(19) United States
(12) Patent Application Publication
(10) Pub. No.: US 2024/0408153 A1
(43) Pub. Date: Dec. 12, 2024

(54) BACTERIA USEFUL IN THE PREVENTION AND/OR TREATMENT OF RESPIRATORY PATHOLOGIES

(71) Applicants: INSTITUT NATIONAL DE RECHERCHE POUR L'AGRICULTURE, L'ALIMENTATION ET L'ENVIRONNEMENT, PARIS (FR); INSTITUT NATIONAL DES SCIENCES ET INDUSTRIES DU VIVANT ET DE L'ENVIRONNEMENT, Palaiseau (FR); UNIVERSITE PARIS-SACLAY, Gif-sur-Yvette (FR)

(72) Inventors: Muriel THOMAS, IGNY (FR); Pascale SERROR, JOUY EN JOSAS (FR); Alexandre DAVID-HACHETTE, SAINTE MAXIME (FR); Jennifer PALOMO, GUYANCOURT (FR)

(21) Appl. No.: 18/701,034

(22) PCT Filed: Oct. 13, 2022
(86) PCT No.: PCT/EP2022/078581

§ 371 Date: Apr. 12, 2024

(30) Foreign Application Priority Data
Oct. 15, 2021 (FR) FR2110971

(51) Int. Cl. A61K 35/744; A61K 45/06; A61P 11/06; A61P 29/00

(52) U.S. Cl. CPC A61K 35/744; A61K 45/06; A61P 11/06; A61P 29/00

(57) ABSTRACT

The invention relates to three bacterial strains belonging to the Enterococcus genus, to a composition comprising at least one of said strains and to the beneficial uses thereof, in particular as a medicament in the treatment and/or prevention of respiratory diseases.

### TECHNICAL FIELD

The invention relates to three bacterial strains belonging to the genus Enterococcus, a composition comprising them, and its use as a medicament, in particular in the treatment and/or prevention of respiratory diseases.

### PRIOR ART

The human body has several microbiota such as intestinal microbiota, oral, cutaneous, vaginal, and more recently pulmonary microbiota has been described. These microbiota gradually set in from birth and orient immune maturation. Colonization by the digestive microbiota has a very significant impact on immunity and health. Since then, numerous studies have described the bacteria present in human microbiota and used them for their beneficial effects on health, such as probiotics.

Enterococcus faecalis (E. faecalis) is one of the enterococci found in the human gastrointestinal tract. In humans, E. faecalis is subdominant in the intestinal microbiota of healthy adults, and dominant in children just after birth. It is a ubiquitous bacterium in humans and animals, found in both the intestine and the oral cavity, as well as in the pulmonary microbiota. However, certain strains of the species are responsible for infections, particularly nosocomial ones.

The species E. faecalis has been described for its immunomodulatory properties, which are important for human beings, particularly in the maturation of colon epithelial cells, protection against relapses of chronic bronchitis, atopic dermatitis and susceptibility to developing asthma. A specific strain has already been described in patent FR 1650656 for its beneficial effects in the treatment and/or prevention of respiratory diseases.

Chronic respiratory diseases such as asthma, chronic obstructive pulmonary disease (COPD), allergic rhinitis, rheumatoid arthritis and inflammatory bowel disease (IBD) affect millions of people worldwide. These chronic diseases have a major impact on the quality of life of patients; no curative treatment for them is available, and their prevalence has increased greatly in Western countries. As a result, these pathologies represent a major medical, social and economic problem worldwide. Current treatments are essentially symptomatic, aiming to treat or possibly prevent attacks occurring periodically, in order to improve patients' quality of life.

Current treatments are based on broad-spectrum immune modulators such as corticosteroids. While these are highly effective as occasional treatments, they can have deleterious side-effects when taken regularly. For example, inhaled corticosteroids can cause growth retardation in children, suppression of the hypothalamic-pituitary-adrenal axis, and an increased risk of osteoporosis. More recently, certain drugs targeting key mediators of inflammation, such as Tumor Necrosis Factor alpha (TNF-α), Interleukin-1 (IL-1) or Interleukin-5 (IL-5) have been developed. However, not all inflammatory diseases respond to the same cytokine inhibitor(s). Finally, some patients are still refractory to existing treatments, whether corticosteroids or specific inhibitors.

There is therefore a strong medical need for new therapeutic solutions that can either treat and/or prevent these conditions, such as respiratory pathologies, or reduce the doses of anti-inflammatory treatments required and/or increase their efficacy.

One aim of the present invention is therefore to provide a simple, more effective and economical alternative solution for preventing and/or treating chronic inflammatory diseases, including respiratory diseases, and also to limit the side effects associated with current treatments, thereby reducing the prevalence of these diseases.

### SUMMARY OF THE INVENTION

To meet this objective, the inventors have identified, among the multitude of bacteria of the species E. faecalis, three new bacterial strains of E. faecalis registered on Jun. 16, 2021 with the Collection Nationale des Cultures de Microorganismes (CNCM), 25 rue du Docteur Roux, 75724 Paris Cedex 15, France under the numbers CNCM I-5699, CNCM I-5700 and CNCM 1-5701, which are particularly useful for reducing inflammation, especially in the prevention and/or treatment of respiratory pathologies.

Indeed, the three strains exhibit an anti-inflammatory effect in vitro on human cells of pulmonary origin (BEAB-2B), but also ex vivo on explants of lungs of axenic mice. In particular, they reduce the release of IL-18, IL-2, IL-5, TNF-α and IL-6. Other strains of E. faecalis tested do not have such properties. The three strains according to the invention also have an anti-inflammatory effect in vivo, in two pre-linear allergic asthma models. They make it possible in particular to reduce eosinophilic recruitment and release of TH2 cytokines into the lungs.

Finally, the strain registered under number CNCM I-5699 also has a protective effect of the respiratory function in the model of asthma in adult mice. The three strains according to the invention thus have quite advantageous properties that are useful in the treatment and/or prevention of respiratory diseases.

Thus, the subject matter of the invention is three strains of the species of E. faecalis registered, respectively, under numbers CNCM I-5699, CNCM I-5700 and CNCM I-5701.

The strains registered under numbers CNCM I-5699, CNCM I-5700 and CNCM I-5701 have a chromosome, respectively, of 2843733 bp (base pair), of 2937166 bp and of 2982831 bp. The chromosome size of strains according to the invention is therefore smaller than that of the strain described in patent FR 1650656 registered under CNCM number 1-4969, whose size is 3056663 bp.

The invention also targets the culture supernatant obtained from at least one of the bacterial strains selected from strains CNCM I-5699, CNCM I-5700 and CNCM I-5701. According to one variant, the supernatant can be obtained from a mixture of strains CNCM 1-5699 and/or CNCM I-5700 and/or CNCM I-5701.

According to another subject matter, the invention also to a composition comprising (i) at least the bacterial strain according to the invention and/or (ii) the supernatant of the culture according to the invention and (iii) at least one acceptable excipient. Said acceptable excipient preferentially being a pharmaceutically acceptable excipient when it is a product intended to be used as a medicament for human use or veterinary use. The composition is then a pharmaceutical composition comprising a strain selected from CNCM I-5699, CNCM 1-5700, CNCM 1-5701 and mixtures thereof and at least one pharmaceutically acceptable excipient.

Finally, the invention is particularly suitable for using said bacterial strain, said supernatant, or said composition according to the invention as a medicament. Indeed, the present invention is particularly suitable for the prevention and/or treatment of inflammatory diseases, in particular respiratory diseases, more particularly chronic diseases such as allergy, asthma, chronic obstructive pulmonary disease (COPD), or allergic rhinitis. Other chronic inflammatory pathologies are also of interest, such as rheumatoid arthritis or chronic inflammatory bowel disease (IBD), or excessive secondary inflammation caused by a primary infection, e.g. bacterial or viral, such as influenza, infant bronchiolitis or SARS.

According to one variant, the strains according to the invention are also useful to prevent and/or treat chronic inflammatory bowel diseases, more preferentially Crohn's disease, or ulcerative colitis.

Other features and advantages will emerge from the detailed description of the invention, examples and figures that follows.

### BRIEF DESCRIPTION OF THE FIGURES

FIG. 1 shows the anti-inflammatory properties of the strains according to the invention in particular the reduction of IL-8 induced by TNF-α in the BEA-2B cells.

FIG. 2 shows the anti-inflammatory properties ex-vivo of the strains according to the invention in particular the reduction of several cytokines responsible for inflammation in axenic mouse lung explants.

FIG. 3 shows the heat map of the strains according to the invention on the production of cytokines by the lung explants.

FIG. 4 shows the protocol of the preclinical model of allergic asthma induced by HDM according to example 3 in mouse pups (Panel A) and in adult mice (Panel B).

FIG. 5 shows the protective effect of the strain registered under number CNCM I-5699 against a decrease in blood oxygenation in a HDM-induced asthma model in adult mice.

FIG. 6 shows the protective effect of the strain registered under number CNCM I-5699 against the increase in pulmonary resistance in a HDM-induced asthma model in adult mice.

### DETAILED DESCRIPTION OF THE INVENTION

### Definition

The term “bacterium” in the sense of the invention is understood to mean a single-cell microorganism capable of being reproduced by cell division. The bacteria are classified by family, genus, species. Each bacterial species comprises a diversity of bacterial strains. The bacterial strains according to the invention belongs to the family Enterococcaceae, the genus Enterococcus and the species faecalis.

Therefore, “bacterial strain” or “strain” within the meaning of the invention is a specific bacterial strain but also all the bacteria derived from the strain or obtained from the strain or corresponding to the bacterial strain and having the same metabolic functions, for example at least one bacterium removed from a colony derived from the strain. Within the meaning the invention, “bacterium (bacteria) according to the invention” is therefore also intended to mean a bacterial strain according to the invention, in particular, the strains registered under numbers CNCM I-5699, CNCM I-5700 and CNCM I-5701 as well as their derived strains.

The term “derived strain” for the purposes of the invention refers to a bacterial strain having a high similarity with at least one of the bacterial strains registered under number CNCM 1-5699, CNCM 1-5700 or CNCM 1-5701. Preferentially, the chromosomal nucleotide sequence of the derived strain comprises a nucleotide sequence having at least 99.9% ANI identity, more preferentially at least 99.91%, at least 99.92%, at least 99.93%, at least 99.94%, at least 99.95%, at least 99.96%, at least 99.97%, at least 99.98%, at least 99.99% ANI identity with the chromosomal nucleotide sequence of at least one of strains CNCM I-5699, CNCM I-5700 or CNCM I-5701.

Within the meaning the invention, “ANI” refers to the percentage of average nucleotide identity calculated from two-by-two comparison of all the chromosomal sequences between the two bacterial strains. According to the general knowledge well known to the person skilled in the art, the genomic DNA can be extracted from said strain of interest registered with e.g. the CNCM from a pure bacterial culture from said strain of interest, followed by DNA sequencing according to various well-known methods, for example Sanger, Roche 454, Illumina, Oxford Nanopore and then the sequenced genome is assembled by biocomputing and the sequences obtained are analyzed. Finally, the chromosomal sequences of interest are compared two-by-two to calculate the ANI.

The term “supernatant” for the purposes of the invention is understood to mean the culture supernatant of the bacterial strain according to the invention optionally comprising cellular components of said strain and/or cell debris of said strain, and/or metabolites and/or molecules secreted by said strain.

“Human” for the purposes of the invention is understood to mean a human patient. Said human patient may belong to any age group, that is, the patient may be an infant, a baby, a child, an adolescent or an adult. It is known that children, babies, infants and elderly people are more sensitive to respiratory diseases, in particular asthma. Preferentially, the invention relates to resistant and/or refractory human patients to corticosteroid treatments.

“Resistant and/or refractory patient to the treatments by corticosteroids” is understood to mean, for the purposes of the invention, a patient who has been given a corticosteroid-based treatment and who does not respond to this treatment, that is to say the patient does not show a reversal of the progression of inflammation or a reversal or regression of the progression of the disease.

“Prevention” for the purposes of the invention is understood to mean the reduction to a lesser degree of the risk or the probability of occurrence of a given phenomenon, that is, in the context of the present invention of the inflammation, preferentially respiratory diseases, e.g. asthma.

“Treatment” for the purposes of the invention is understood to mean reducing the progression of the disease, stabilizing, reversing or regressing, or even interrupting or inhibiting the progression of inflammation, preferentially of respiratory illnesses, such as asthma. In the context of the invention, these terms also apply to one or more symptoms of said diseases of the present invention.

For the purposes of the invention, “respiratory disease” refers to diseases of the respiratory system or diseases causing respiratory disorders.

For the purposes of the invention, the term “acceptable excipient” is intended to mean any compound making it possible to facilitate the formulation of the composition and not modifying the nature of the biological activity of the active ingredient. An acceptable excipient may be a solvent, buffer, saline solution, plasticizer, lubricant, dispersion medium, absorption retarding agent, flow agent, isotonic agent. Preferentially, it may be pharmaceutically acceptable excipients that are selected according to the pharmaceutical form and the desired mode of administration, among the usual excipients known to the person skilled in the art and suitable for human and/or veterinary use. The excipient will thus be chosen as a function of the administration route, for example suitable for oral, nasal, intravenous, intramuscular, topical administration etc.

### Bacterial Strain or Supernatant According to the Invention

The present invention therefore relates to a bacterial strain of E. faecalis selected from the strains registered under numbers CNCM I-5699, CNCM I-5700, CNCM I-5701 and their mixture.

The inventors have discovered and characterized these E. faecalis bacterial strains from human donors, or from cheeses, and have demonstrated, in in vitro models, anti-inflammatory effects, in particular the reduction of IL-8 production induced by a pro-inflammatory molecule, TNF-α.

The anti-inflammatory properties of the strains according to the invention were also characterized on a model of lung explants that is closer to the pulmonary physiology than the existing cell lines. The various strains were thus coincubated with explants of lungs of axenic mice in culture ex vivo and also have an anti-inflammatory effect, in particular they reduce IL-18, IL-2, IL-5, TNF-α or IL-6 release.

These initial results, in particular on axenic mouse lung explants, confirm the clinical benefit of the strains according to the invention as a medicament, particularly in the prevention and/or treatment of respiratory diseases.

Thus, the invention relates at least to a bacterial strain of E. faecalis selected from the strains registered under numbers CNCM I-5699, CNCM I-5700 and CNCM I-5701.

The sequence of the 16S RNA gene of the CNCM I-5699 strain, the CNCM I-5701 strain and the CNCM I-5700 strain are identical and SEQ ID NO: 1.

16S ribosomal RNA (16S rRNA) is ribosomal RNA constituting the small subunit of prokaryotic ribosomes. The genes coding for this RNA are called 16S rDNA. The 16S rDNA sequence is very widely used in phylogenesis due to its very conserved structure, which makes it possible to reconstruct the evolutionary history of organisms and in particular of prokaryotes and bacteria.

However, the 16S rDNA sequence does not always allow differentiation of two strains of the same species having different properties, for example anti-inflammatory properties, such as a different cytokine profile. This results in a different efficacy for each strain. Therefore, the person skilled in the art, given their general knowledge, is able to characterize a bacterial strain according to other parameters such as the calculation of phylogenetic distance based on the nucleotide sequence of the chromosome (ANI) or its size.

Thus, strains CNCM I-5699, CNCM I-5700 and CNCM I-5701 possess chromosomes whose respective sizes are 2843733 bp, 2937166 bp and 2982831 bp. The chromosome size of strains according to the invention is therefore smaller than that of the strain described in FR 1650656 registered under CNCM number 1-4969, whose size is 3056663 bp.

Moreover, the phylogenetic distance based on the chromosome sequence (ANI) was also calculated. The results are presented in table 1 below.

TABLE 1

| | CNCM | CNCM | CNCM |
|---|---|---|---|
| ANI | I-4969 | I-5699 | I-5700 |
| CNCM I-5699 | 99.04% | | |
| CNCM I-5700 | 99.87% | 99.17% | |
| CNCM I-5701 | 99.78% | 99.14% | 99.79% |

The invention also relates to strains derived from at least one of the bacterial strains registered under numbers CNCM I-5699, CNCM 1-5700 or CNCM 1-5701 which make it possible to maintain or improve the described capacities of the strains according to the invention. Said derived strains can thus be produced naturally or intentionally by mutagenesis methods known from the prior art. By way of example, the mutagenesis methods that can be implemented within the scope of the present invention are the growth of the original microorganism in the presence of mutagenic or stress-producing agents, or by genetic engineering aiming to modify specific or non-specific genes such as directed mutagenesis or random mutagenesis.

Preferentially, said derived strain comprises a nucleotide sequence having at least 99.9% ANI (Average Nucleotide Identity) identity with the chromosomal nucleotide sequence of one of the bacterial strains registered under the numbers CNCM 1-5699 or CNCM 1-5700 or CNCM I-5701. More preferentially, the derived strain comprises a nucleotide sequence having at least 99.91%, at least 99.92%, at least 99.93%, at least 99.94%, at least 99.95%, at least 99.96%, at least 99.97%, at least 99.98%, at least 99.99% ANI identity with the chromosomal nucleotide sequence of at least one of strains CNCM I-5699, CNCM 1-5700 or CNCM I-5701.

According to a particularly preferred embodiment, the invention relates to the strain of E. faecalis registered under number CNCM I-5699.

The strains according to the invention can be produced by culturing, for example, in a growth medium known to the person skilled in the art (for example, a BHI medium: “Brain-Heart Infusion”) of 8 hours to 3 days, at a temperature of 30-37° C., with or without adjustment of the pH. The fermentation broth containing the bacterial cells is collected. The broth can be used as-is, concentrated or freeze-dried. Advantageously, the bacteria will be collected, for example by centrifugation and then resuspended in an appropriate buffer, for example PBS (“phosphate buffered saline”). The bacterial concentration can be established using a flow cytometer or another equivalent method. The strain according to the invention is particularly advantageous in that it leads to a reduction in inflammation. Thus, the bacterium of the invention makes it possible to attenuate the effects of inflammatory, in particular respiratory, diseases.

A further object of the invention relates to the culture supernatant obtained from one of the bacterial strains according to the invention, but also a strain derived therefrom. Preferentially, the culture supernatant comprises at least one of the constituents selected from a bacterial cell compound, a bacterial cell debris, a metabolite and/or molecule(s) secreted by the strain and/or the derived strain, or a mixture thereof.

Thus, the cell compounds and/or cell debris may be components of the wall, nucleic acids, membrane components, proteins, lipids, etc. The secreted metabolites or molecules may be any molecule produced or modified by the bacterium due to its metabolic activity during its growth, its use in technological processes (for example, but not limited thereto, methods for producing food or medicaments). By way of example, the metabolites or molecules may be proteins, amino acids, enzymes, lipids, nucleic acids, etc. “Metabolite and/or molecule(s) secreted by the strain and/or the derived strain” means a molecule produced and exported or released outside the bacterium by the bacterium. Thus, the invention also relates to the culture supernatant obtained from one of the bacterial strains according to the invention or mixtures thereof.

### Composition

The strain(s) according to the invention or derived strain(s) and/or the culture supernatant described previously and the bacteria derived from said strains are advantageously administered in a composition.

Thus, the invention relates to a composition comprising: (i) at least one bacterial strain according to the invention and/or (ii) a supernatant according to the invention and (iii) at least one acceptable excipient.

The composition may also comprise a physiologically acceptable medium, that is, a medium that is compatible with the body of the individual to which said composition is to be administered. It may involve, for example, a non-toxic solvent such as water, buffer, saline solutions. In particular, said medium is compatible with oral administration.

Thus, preferentially, the composition comprises at least one bacterial strain selected from one of the strains registered under number CNCM I-5699, number CNCM I-5700, number CNCM I-5701 and mixtures thereof; and/or the culture supernatant obtained from said strains and at least one acceptable excipient and/or a physiologically acceptable medium.

According to a particularly preferred embodiment, the composition comprises the strain registered under number CNCM I-5699.

According to one variant, the composition comprises the strain registered under number CNCM I-5699 and the strain registered under number CNCM I-5700 and the strain registered under number CNCM I-5701.

The bacterial strain according to the invention may be present in any form that produces the expected effects and efficacy described in the present application. In particular, the strain can be in living (cultivable or not), dead, semi-live, attenuated or inactivated form. The dead, semi-living, attenuated, or inactivated form induces the same effects as the live strain and therefore also has anti-inflammatory properties, making it possible in particular to prevent and/or treat respiratory diseases.

These shapes can be obtained by various techniques well known to the person skilled in the art. Mention will be made, for example, of the following techniques: irradiation, thermal inactivation or freeze-drying, in particular by heat, exposure to an appropriate pH, UV, gamma rays, X-rays or high pressure.

The term “semi-living” refers to a bacterium with a low physiological activity whose ability to proliferate is reduced, temporarily or definitively. The term “inactivated” refers to a bacterium which is no longer capable, temporarily or definitively, of proliferating. The term “dead” refers to a bacterium that is definitively no longer capable of proliferating. The dead or inactivated bacteria can have intact or broken cell membranes. Thus, the term “inactivated” also refers to the obtained extracts and lysates of bacteria.

It is particularly advantageous to use such shapes in the pharmaceutical compositions of the invention.

The composition according to the invention can also comprise at least one additional compound. The additional compound may be an ingredient, a molecule, an active principle, a microorganism, a bacterium or a mixture of bacteria. Preferably, the additional compound is an anti-inflammatory agent, more preferentially a corticosteroid, even more preferentially budesonide. According to one variant, the additional compound may be a probiotic and/or a prebiotic.

The composition according to the invention is in any form acceptable to be administered to a patient, preferentially a human patient or (non-human) animal. Thus, the composition according to the invention also relates to veterinary uses.

Preferably, the composition according to the invention is in solid, liquid or lyophilized form.

When the composition is in liquid form, it may in particular comprise bacterial strains according to the invention and/or of the derived strain and/or a culture medium physiological acceptable to said bacteria which makes it possible to maintain them, such as for example the medium BHI, or an equivalent medium not containing any derived product of animal origin.

When the composition according to the invention is in solid form, the bacterial strains according to the invention and/or of the derived strain can be present in lyophilized form, and may also comprise excipients such as, for example, microcrystalline cellulose, lactose, sucrose, fructose, levulose, starches, stachyose, raffinose, amylum, calcium lactate, magnesium sulfate, sodium citrate, calcium stearate, polyvinylpyrrolidone, maltodextrin, galactooligosaccharides, fructooligosaccharides, pectins, beta-glucans, lactoglobulins, isomaltooligosaccharides, polydextroses, mannitol, sorbitol, glycerol, silica dioxide, magnesium stearate, cysteine, mannose, galactose, anhydrous glucose, glucose monohydrate, and/or mixtures thereof.

The person skilled in the art will know how best to choose the routes and modes of administration of the composition according to the invention, as well as the optimal dosage and galenic forms, according to the criteria generally taken into account in the manufacture of a medicinal product or the establishment of a pharmaceutical or veterinary treatment. Preferably, the composition according to the invention is in a form suitable for oral, nasal, parenteral, rectal, sublingual, ocular, atrial, intramuscular, intravenous, inhaled or cutaneous administration, more preferentially nasal or inhaled, using a nebulizer device.

The composition may be in any suitable form. Thus, the composition according to the invention may be in a form chosen from a powder, powder to be nebulized, microencapsulated powder, microencapsulated powder to be nebulized, gelatin capsule, capsule, tablet, pellet, granules, solution, solution to be nebulized, emulsion, emulsion to be nebulized, suspension, suspension to be nebulized, ampoule, suppository, inhaler and syrup, more preferentially in the form of an inhaler such as a nebulizer apparatus.

The composition according to the invention may potentially be in gastro-resistant form, for example a coated tablet containing microencapsulated bacteria.

Particularly preferably, the composition comprises at least 10³ colony forming units (CFU) of bacteria per daily dose of composition to be administered, preferentially 10⁴ to 10⁹ CFU, more preferentially of 10⁵ to 10⁷ CFU.

Preferentially, this corresponds to a daily dose of bacteria to be administered, regardless of the weight of the person or of the animal. Thus and preferably, this dose is administered all at once. The useful composition then comprises at least 10³, preferentially 10⁴ to 10⁹ CFU, even more preferentially 10⁵ to 10⁷ CFU of bacteria per daily dose to be administered.

Said bacteria are a mixture of bacteria corresponding or derived from one of the strains according to the invention, including the derived strains.

The term CFU (Colony Forming Unit) is a unit making it possible to count the number of bacteria capable of giving rise to a colony during propagation, that is to say viable bacteria. It should be understood that non-viable bacteria can also be present in the compositions and that in general they should not have a negative effect on the properties of the living bacteria of the composition.

Preferentially, the daily dose is measured per gram or milliliter of the final composition according to the invention.

According to another preferred object, when the composition according to the invention comprises a mixture of bacteria, living or not, said mixture comprising at least living bacteria, the composition preferentially comprises at least 1% of living bacteria (in number), more preferentially at least 10% living bacteria (in number), even more preferentially at least 50% living bacteria (in number).

The living bacteria are a mixture of bacteria corresponding to one of the strains according to the invention, including the derived strains.

In the case of the implementation of a supernatant of any one of the aforementioned bacterial strains, the composition according to the invention including said supernatant may in particular comprise a content thereof of between 0.1 and 99% by weight, namely from 5 to 95% by weight, in particular from 10 to 90% by weight and more particularly from 15 to 85% by weight, relative to the total weight of the composition.

Thus, a composition according to the invention may comprise a supernatant content of between 0.1 and 99% by weight, namely from 5 to 95%, in particular from 10 to 90% by weight, more particularly from 15 to 85% by weight, relative to the total weight of the composition.

When the composition is intended for therapeutic use, the composition comprises at least one pharmaceutically acceptable excipient, the composition according to the invention is then a pharmaceutical composition.

The term “pharmaceutically acceptable excipient” here means an excipient whose administration to an individual is not accompanied by significant harmful effects. The pharmaceutically acceptable excipients are well known to the person skilled in the art.

Thus, the invention also relates to a pharmaceutical composition comprising the ingredient(s) included in the composition according to the invention and according to one of any embodiments described above. Said pharmaceutical composition may be a medicament.

According to another aspect, the composition according to the invention may be in the form of a food product, a beverage, a nutraceutical, a food additive, a food supplement or a dairy product.

When the composition according to the invention is comprised in a food supplement for oral administration, the latter may be in the form capsules, gel capsules, soft capsules, tablets, sugar-coated tablets, pills, pastes, pellets, gums, drinkable solutions or emulsions, a syrup or a gel.

A food supplement according to the present invention may further comprise a sweetener, a stabilizer, an antioxidant, an additive, a flavoring agent and/or a colorant. The formulation of the latter is carried out by means of the usual methods for producing sugar-coated tablets, gel capsules, gels, hydrogels for controlled release, emulsions, tablets or capsules.

A composition according to the present invention may also be in the form of a nutritional composition. Such nutritional composition according to the present invention may be in the form of a yogurt, a fermented food, a cereal bar, breakfast cereals, a dessert, a refrigerated food, a frozen food, a soup, a pet food, a liquid suspension, a powder, a tablet, a gum or a sweet.

A nutritional composition according to the present invention may further comprise at least one ingredient selected from: antioxidants, fish oils, vegetable oils, DHA, EPA, vitamins, minerals, phytonutrients, protein, lipid, probiotics, prebiotics, postbiotics, and combinations thereof.

### Use

The composition according to the invention is useful as a medicament for use in human or veterinary health.

Preferentially, the composition according to the invention is intended to prevent and/or treat inflammation and thus be used in the treatment and/or prevention of inflammatory diseases, more preferentially respiratory diseases and/or inflammatory diseases of the intestine and/or rheumatoid arthritis and/or inflammatory diseases associated with a viral or bacterial infection.

Examples include asthma (mild, moderate or severe), e.g. bronchial, allergic, intrinsic, extrinsic, exercise-induced, drug-induced (including aspirin and non-steroidal anti-inflammatory drugs-NSAIDs) and dust-induced asthma, steroid-resistant asthma, bronchitis, including infectious and eosinophilic bronchitis, chronic obstructive pulmonary diseases (COPD), such as COPD (chronic obstructive pulmonary disease), cystic fibrosis, pulmonary fibrosis, including cryptogenic fibrosing alveolitis, idiopathic pulmonary fibrosis, idiopathic interstitial pneumonias, fibrosis complicating antineoplastic therapy and chronic infection, including tuberculosis and aspergillosis and other fungal infections; complications of lung transplantation; vasculitis and thrombotic disorders of the pulmonary vasculature and pulmonary hypertension (including pulmonary arterial hypertension); antitussive activity, including treatment of chronic cough associated with inflammatory and secretory disorders of the respiratory tract, and iatrogenic cough; acute and chronic rhinitis, including rhinitis medicamentosa, and vasomotor rhinitis; perennial and seasonal allergic rhinitis, including nervous rhinitis (hay fever); nasal polyposis; acute viral infection, including the common cold, and infection due to respiratory syncytial virus (RSV), influenza, pulmonary bronchopathies, coronavirus infections (notably SARS) and adenovirus, pulmonary edema, pulmonary embolism, pneumonia, pulmonary sarcoidosis, silicosis, farmer's lung and related diseases; hypersensitivity pneumonitis, respiratory failure, acute respiratory distress syndrome, emphysema, chronic bronchitis, tuberculosis and lung cancer.

The composition according to the invention also relates to respiratory pathologies having “immune disorders” in common with those observed in asthma such as inducing certain cytokines (IL-6, TSLP, IL-8, IL-5, IL-13, IL-17) and mucosal deregulations (hyperproduction of mucus).

In particular, the uses and compositions of the present invention encompass the prevention and treatment of chronic respiratory diseases, for example asthma, COPD and rhinitis. Even more particularly, the uses and compositions of the present invention relate to the prevention and treatment of asthma.

The inventors have demonstrated that the strains according to the invention are particularly effective for preventing asthma when they are administered before the appearance of the set of symptoms of the disease. Thus, the use of the strain of the invention and the composition comprising it may in particular be used effectively to prevent the appearance of asthma in patients known for their predisposition for this pathology (for example, patients who have a family predisposition to the development of asthma).

Given the common inflammatory routes, the composition according to the invention is also useful in a disease selected from Crohn's disease, ulcerative colitis, diverticulitis, esophagitis, gastritis, pancreatitis, peptic ulcers, irritable bowel syndrome.

Finally, the composition according to the invention is particularly of interest when humans or animals are resistant and/or refractory to corticosteroids.

The invention is now illustrated by non-limiting examples of compositions according to the invention and by results.

### EXAMPLES

### Example 1—Anti-Inflammatory Effect of the Strains According to the Invention

BEAS-2B cells were co-incubated with TNF-α (1 pg/ml) alone or with bacteria E. faecalis according to the invention (CNCM I-5699, CNCM 1-5700, CNCM 1-5701) or outside the invention (CNCM I-4969, E. faecalis 1 or E. faecalis 2) MOI 250:1. After 6 h of incubation, the IL-8 concentration was measured in the supernatant. The IL-8 levels are expressed as a percentage of the IL-8 level induced by a stimulation of TNF-α of 6 h. The results correspond to the median of 5 experiments, at least n=3-6 technical replicates per condition. *p<0.05 compared with the TNF-α control.

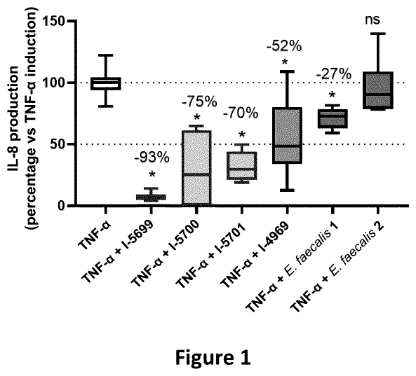

The inventors observed that the three strains of E. faecalis according to the invention (CNCM I-5699, CNCM I-5700, CNCM I-5701) have a strong “anti-IL-8” effect, respectively 93%, 75% and 70% compared to the strain CNCM I-4969 and to two other strains of E. faecalis. The CNCM I-4969 strain certainly has an effect, but lower than the three strains according to the invention. Thus, the strains E. faecalis according to the invention have useful anti-inflammatory properties (FIG. 1).

### Example 2—Anti-Inflammatory Effect Ex-Vivo of the Strains According to the Invention

The inventors continued their work by comparing the 3 strains according to the invention with 5 other strains of E. faecalis (CNCM I-4969, E. faecalis 1, E. faecalis 2, E. faecalis 3, E. faecalis 4).

The lung explants, or PCLS (Precision-Cut Lung Slices), are culture sections of 200-250 μm, of lungs originating from axenic mice. The explants are optionally coincubated with 50 CFU (colony forming units) of bacteria: strains of E. faecalis. After 24 h, the culture supernatants are taken and the amount of cytokines measured by Luminex. The results represent the percentage of cytokine measured, compared with the condition of the medium alone. The data are grouped together by 4 experiments, at least n=6 biological replicates per condition. *p<0.05 compared with the medium control.

The inventors thus characterized the anti-inflammatory effects on a model of lung explants that is closer to pulmonary physiology in comparison with cell lines. The various strains of E. faecalis were co-incubated with explants of lungs of axenic mice in culture ex vivo. These explants have a basal production of different cytokines, which can be modified by the presence of immunomodulatory elements.

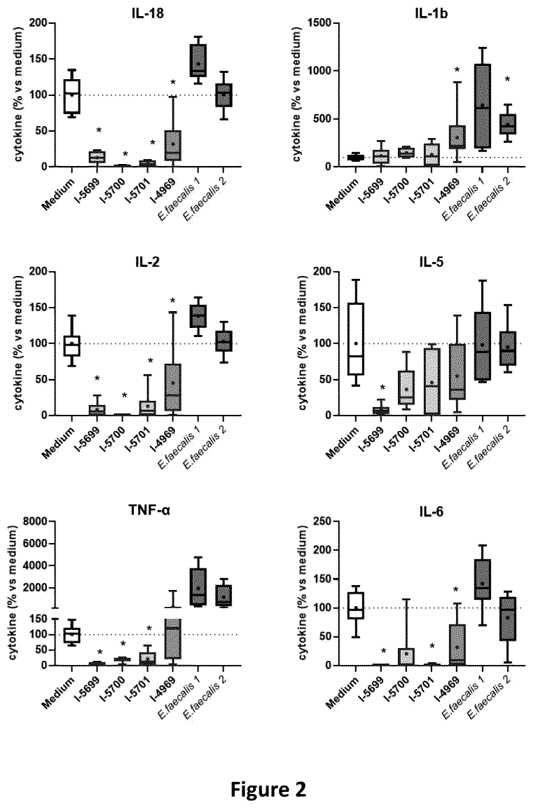

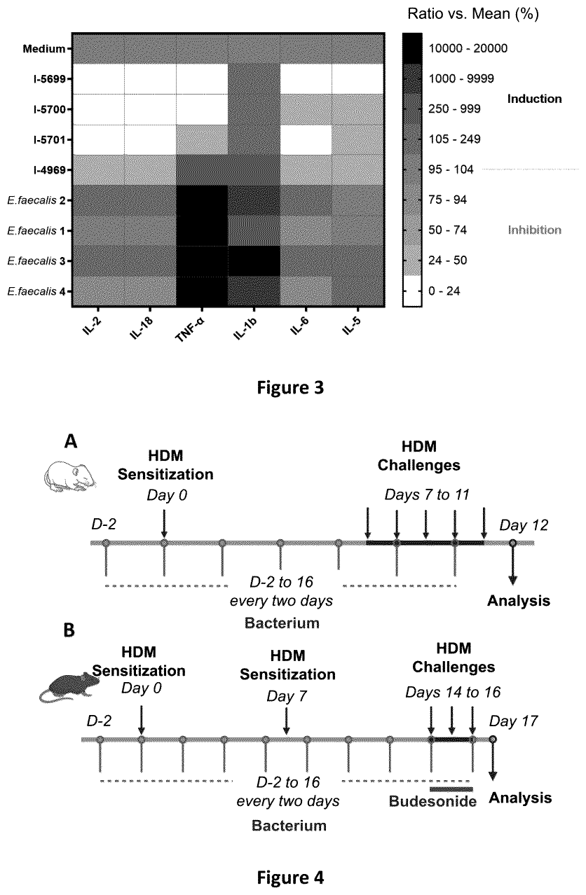

The inventors demonstrated that the 3 strains of E. faecalis according to the invention has an anti-inflammatory effect on mouse lung explants. In particular, they reduce the release of IL-18, IL-2, IL-5, TNF-α and IL-6 (FIGS. 2 and 3). The CNCM I-4969 strain has a significantly lower effect than the three strains according to the invention and the 4 other strains tested (E. faecalis 1, E. faecalis 2, E. faecalis 3, E. faecalis 4) have neutral or pro-inflammatory effects (FIG. 3).)

### Example 3—Study on Preclinical Models of HDM-Induced Asthma

The protocol of the preclinical model of allergic asthma induced by HDM in mouse pups is shown in FIG. 4, panel A.

Mouse pups were sensitized by intranasal administration of HDM (House Dust Mite) (1 μg) on DO (Sensitization) then at 10 μg of D7 at D11 (Challenges). The strains according to the invention were administered intranasally to 10⁶ CFU every 2 days from D-2 to D10 (FIG. 4 Panel A). At D12, the inventors analyzed growth monitoring, carried out bronchoalveolar lavage (BAL) and analyzed the cells and cytokines of the lung tissue.

The protocol of the preclinical model of allergic asthma induced by HDM in adult mice is shown in FIG. 4, panel B.

Adult mice were also sensitized by intranasal administration of HDM (House Dust Mite) (25 μg) on DO and D7 (Sensitization) then at 10 μg of D14 at D16 (Challenges). The strains according to the invention were administered intranasally to 10⁶ CFU every 2 days from D-2 to D16. In one control group, budesonide was administered intranasally (3 mg/kg) from D14 to D16 (FIG. 4 Panel B). At D17, the inventors analyzed growth monitoring, carried out bronchoalveolar lavage of the cells and analyzed the cytokines found in the lavage and lung tissue.

The results are shown in Table 2 below.

TABLE 2
Analysis of the mouse pups

| | | | | | | TH2 | | | | TH17 | | | PI | | |
|---|---|---|---|---|---|---|---|---|---|---|---|---|---|---|---|
| | | | | | | cytokines | | | | cytokines | | | cytokines | | |
| | number | growth | BAL cells | | | (lungs) | | | | (lungs) | | | (lungs) | | |
| | of | retard- | | eosino | neutral | IL- | IL- | IL- | IL- | IL- | IL- | IL- | IFN- | IL- | IL- |
| Strains | mice | ation | total | (nb) | (nb) | 5 | 4 | 13 | 33 | 17A | 22 | 23 | γ | 6 | 18 |
| CNCM | 16 | 1 | 2 | 2 | NA | 2 | 2 | 1 | 1 | 2 | 2 | 2 | 2 | 2 | 2 |
| I-4969 | | | | | | | | | | | | | | | |
| CNCM | 8 | 1 | 2 | 2 | NA | 2 | 2 | 1 | 1 | 2 | 2 | 2 | 1 | 2 | 2 |
| I-5699 | | | | | | | | | | | | | | | |
| CNCM | 8 | 1 | 2 | 2 | NA | 1 | 2 | 0 | 1 | 0 | −1 | 1 | 2 | 2 | 1 |
| I-5700 | | | | | | | | | | | | | | | |
| CNCM | 8 | 1 | 1 | 2 | NA | −1 | −1 | −1 | 0 | −1 | −2 | −1 | 0 | 0 | −1 |
| I-5701 | | | | | | | | | | | | | | | |

| | Adult model analyses | | | | | | | | | | | | | | |
|---|---|---|---|---|---|---|---|---|---|---|---|---|---|---|---|
| | | | | | | TH2 cytokines | | | | TH2 cytokines | | | | | |
| | number | | BAL cells | | | (lungs) | | | | (BAL) | | | | histology | |
| | of | weight | | eosino | neutral | IL- | IL- | IL- | IFN- | IL- | IL- | IL- | IFN- | | In- |
| Strains | mice | change | total | (nb) | (nb) | 5 | 4 | 13* | γ* | 5 | 4 | 13 | γ | mucus | filtration |
| CNCM | 10 | 0 | 2 | −2 | −1 | 1 | −1 | −1 | −1 | 1 | 1 | 1 | 1 | 0 | 0 |
| I-4969 | | | | | | | | | | | | | | | |
| CNCM | 10 | 0 | 0 | 1 | 0 | 2 | 2 | −1 | −1 | 2 | 1 | 2 | 2 | 0 | 1 |
| I-5699 | | | | | | | | | | | | | | | |
| CNCM | 10 | 0 | 1 | 1 | 0 | 2 | 2 | −1 | −2 | 2 | 2 | 2 | 2 | 1 | 1 |
| I-5700 | | | | | | | | | | | | | | | |
| CNCM | 10 | 0 | 1 | 2 | −1 | 2 | 2 | −2 | −2 | 2 | 2 | 2 | 2 | 1 | 1 |
| I-5701 | | | | | | | | | | | | | | | |
| Ctr | 10 | −2 | 2 | 2 | 0 | 2 | 2 | −1 | −1 | 2 | 2 | 2 | 2 | 1 | 2 |
| Budesonide | | | | | | | | | | | | | | | |

LEGEND

| Score | Strain's effect on the modification of the criterion by HDM |
|---|---|
| 2 | strong protection (>50%) |
| 1 | protection (10%-50%) |
| 0 | no visible effect |
| −1 | worsening (10%-50%) |
| −2 | strong worsening (>50%) |

underlined: p < 0.05

The immune cells found in BAL, the TH2 cytokines present in the lungs and the BAL, as well as the histopathology are markers of the development of asthma common to mouse models of asthma and human patients. The data in Table 2 come from an experiment with 8 and 10 animals per group, in mouse pups and in adults, respectively. They indicate the protective (positive values) or aggravating (negative values) effects of administering the strains of E. faecalis according to the invention (CNCM I-5699, CNCM I-5700, CNCM I-5701) and outside the invention (CNCM I-4969) in comparison with the groups of animals treated only with HDM. In the protocol used on adult mice, the effects were compared with those of budesonide, a clinical reference corticosteroid. In the protocol in mouse pups, this comparison was not done.

The inventors thus shown that the intranasal administration of the strains of E. faecalis according to the invention has an improved anti-inflammatory effect compared to the CNCM I-4969 strain in 2 preclinical models of asthma in newborn and adult mouse. They reduce the recruitment of cells found in BAL, as well as the release of pro-inflammatory cytokines in the lungs, in particular IL-5, IL-4, IL-13 and IFN-γ.

### Example 4—Effect of the CNCM 1-5699 Strain on the Respiratory Functions of Adult Mice in a Preclinical HDM-Induced Asthma Model

Adult mice were sensitized by intranasal administration of HDM (25 μg) at D0 and D7 and then at 10 μg of D14 to D16 (FIG. 4 Panel B). The strain CNCM I-5699 was administered intranasally at 10⁶ CFU every 2 days from D-2 to D16. Budesonide was administered intranasally (3 mg/kg) from D14 to D16. The pulmonary function was measured every two days using a pulse oximeter at D-2 before the first treatment, then every 2 days until the end of the experiment. The measurement makes it possible to obtain the blood oxygen saturation level, which can be affected in the event of alteration of the respiratory function, in particular during the development of asthma. At D17, the inventors measured the pulmonary dynamic resistance. A similar test is used in humans to diagnose bronchial hyperreactivity (BHR), which is particularly observed in asthma, and which is manifested by an increase in pulmonary resistance. The general principle of this bronchial challenge test consists of the inhalation of increasing doses of methacholine, a substance which indirectly induces contraction of the bronchial smooth muscle, resulting in changes in airway caliber, an increase in airway resistance and a decrease in gas flow through the airway. The mice were anesthetized and intubated. Increasing concentrations of an aerosol methacholine were administered. The resistance was recorded with a plethysmograph.

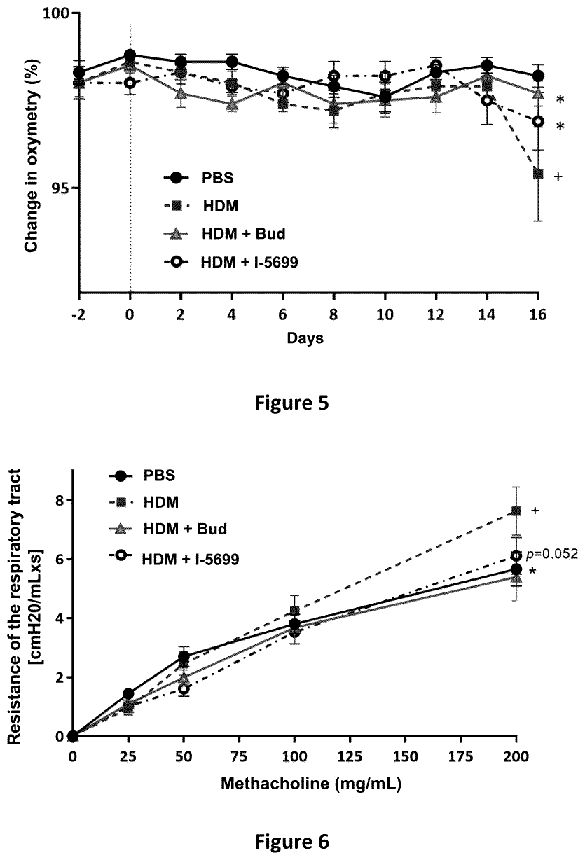

The results are shown in FIGS. 5 and 6.

Allergic asthma was induced in naïve C57BL/6J mice by HDM intranasal administrations (FIG. 4 Panel B). The CNCM I-5699 strain was administered every two days intranasally at a rate of 1.10⁶ CFU/mouse from D-2 to D16. Pulse oximetry was measured every two days from D-2 to D16. The data are represented in the form of mean±SEM of 10 mice per group. After statistical evaluation of the differences between the experimental groups, the p values less than 0.05 were considered to be statistically significant: +p<0.05, PBS group compared to the HDM group; *p<0.05 HDM group compared to the treated groups (FIG. 5).

Allergic asthma was induced in naïve C57BL/6J mice by HDM intranasal administrations (FIG. 4 Panel B). The CNCM I-5699 strain was administered every two days intranasally at a rate of 1.10⁶ CFU/mouse from D-2 to D16. The mice were euthanized at D17, 24 hours after the last challenge, and the resistance of the respiratory tract was measured with subtraction of the PBS baseline. The data are represented as mean±SEM of 10 mice per group. After statistical evaluation of the differences between the experimental groups, the p values less than 0.05 were considered to be statistically significant: +p<0.05, PBS group compared to the HDM group; *p<0.05 HDM group compared to the treated groups (FIG. 6).

The inventors thus shown that the intranasal administration of the strain of E. faecalis CNCM I-5699 according to the invention has a protective effect of the respiratory function in a preclinical model of asthma in mice. They observed better blood oxygenation (FIG. 5) and better lung contractibility (FIG. 6) in the presence of the CNCM I-5699 strain, in a preclinical model of asthma induced by HDM.

1. A bacterial strain of the species of Enterococcus faecalis registered under number CNCM I-5699 or CNCM I-5700 or CNCM I-5701.

2. The bacterial strain according to claim 1, characterized in that the strain is the strain of Enterococcus faecalis registered under number CNCM I-5699.

3. A culture supernatant obtained from the bacterial strain according to claim 1.

4. A composition comprising (i) at least one bacterial strain according to claim 1 and/or (ii) a supernatant of the composition and (iii) at least one acceptable excipient.

5. The composition according to claim 4, characterized in that it comprises the three strains of Enterococcus faecalis registered respectively under numbers CNCM I-5699 or CNCM I-5700 or CNCM I-5701.

6. The composition according to claim 4, characterized in that the bacterium is living, dead, attenuated or inactivated.

7. The composition according to claim 4, characterized in that it is in solid, liquid or freeze-dried form.

8. The composition according to claim 4, characterized in that the living bacteria represent at least 10³ CFU.

9. The composition according to claim 4, characterized in that said composition also comprises an anti-inflammatory agent, preferentially a corticosteroid.

10. The composition according to claim 4, for use thereof as a drug for humans or animals.

11. The composition according to claim 10, for its use in the prevention and/or treatment of respiratory diseases and/or inflammatory bowel diseases and/or rheumatoid arthritis and/or inflammatory diseases associated with a viral or bacterial infection.

12. The composition according to claim 11, for its use in the prevention and/or treatment of a disease selected from asthma, bronchitis, bronchiolitis, COPD, pulmonary fibrosis, rhinitis.

13. The composition according to claim 10, characterized in that the human or animal is resistant to corticosteroids.


SEQUENCE LISTING (1 sequence)

SEQ ID NO: 1 - DNA, 1522 bp, Enterococcus faecalis
  source 1..1522: mol_type = other DNA
agagtttgatcctggctcaggacgaacgctggcggcgtgcctaatacatgcaagtcgaacgcttctttcctcccgagtgcttgcactcaattggaaagaggagtggcggacgggtgagtaacacgtgggtaacctacccatcagagggggataacacttggaaacaggtgctaataccgcataacagtttatgccgcatggcataagagtgaaaggcgctttcgggtgtcgctgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggacgttagtaactgaacgtcccctgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgctagaccgcgaggtcatgcaaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattggggtgaagtcgtaacaaggtagcc